(19) United States
(12) Patent Application Publication
(10) Pub. No.: US 2022/0186286 A1
(43) Pub. Date: Jun. 16, 2022

(54) FLUORESCENT PROBE FOR DETECTING ACTIVATION OF SIALIDASE

(71) Applicant: The University of Tokyo, Tokyo (JP)

(72) Inventors: Yasuteru URANO, Toyota (JP); Mako KAMIYA, Tokyo (JP); Charlotte RIVAS, Tokyo (JP)

(21) Appl. No.: 17/593,009

(22) PCT Filed: Mar. 5, 2020
(86) PCT No.: PCT/JP2020/009391

§ 371 Date: Sep. 3, 2021

(30) Foreign Application Priority Data
Mar. 5, 2019 (JP) 2019-039095

(51) Int. Cl. C12Q 1/40; C07H 17/04

(52) U.S. Cl. CPC C12Q 1/40; G01N 2021/7786; C07H 17/04

(57) ABSTRACT

A fluorescent probe for sialidase activity detection, is a compound represented by the following general formula or a salt thereof.

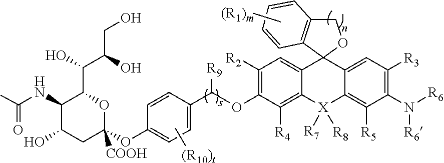

R₁, if present, represents the same or different monovalent substituent present on a benzene ring. R₂ and R₃ each independently represent a hydrogen atom, an alkyl group having 1 to 6 carbon atoms, or a halogen atom. R₄ and R₅ each independently represent a hydrogen atom, an alkyl group having 1 to 6 carbon atoms, or a halogen atom. R₆ represents a hydrogen atom, an alkyl group having 1 to 5 carbon atoms, or a fluorinated alkyl group having 1 to 5 carbon atoms. R₆′ represents a hydrogen atom or an alkyl group having 1 to 6 carbon atoms, and R₆′ may form, together with R₃ or R₅ a five to seven-membered heterocyclyl or heteroaryl containing a nitrogen atom to which R₆′ is bonded.

### TECHNICAL FIELD

The present invention relates to a novel fluorescent probe for detecting sialidase activity.

### BACKGROUND ART

Sialidases (neuraminidases) are an exo glycolytic enzyme that liberates a sialic acid from the non-reducing end of a sugar chain, and have a role in important cellular functions such as cell proliferation/differentiation and apoptosis. Furthermore, a sialidase is present on the surface of an influenza virus and has a role in virus proliferation. Therefore, sialidases can be a biomarker for detection of a disease state or viral infection. However, some existing fluorescent probes for sialidase activity detection function in an ultraviolet region, and some have a property such that the enzymatic reaction product precipitates. Thus, all of the existing fluorescent probes for sialidase activity detection have a problem of poor biocompatibility.

For example, 2′-(4-methylumbelliferyl)-α-D-N-acetylneuraminic acid (4MU-NANA) is an artificial sialidase substrate that is widely used for fluorescence detection of a sialidase. This probe is excited and emits light at high energy wavelengths, and therefore is rather not suitable for in vivo fluorescence imaging. In 2014, Suzuki et al. reported a novel fluorescent sialidase probe having an improved fluorescence property (BTP3-Neu5Ac), but this probe is only suitable for histochemical staining because its hydrolysis product is insoluble in water (Non Patent Literature 4). Therefore, a fluorescent sialidase substrate for in-vivo imaging is urgently awaited.

### CITATION LIST

### Non Patent Literature

Non Patent Literature 1: J. Ferlay, I. Soerjomataram, R. Dikshit, S. Eser, C. Mathers, M. Rebelo, D. M. Parkin, D. Forman, F. Bray, Int. J. Cancer, 2015, 136, E 359.

Non Patent Literature 2: J. Vansteenkiste, L. Crino, C. Dooms, J. Y. Douillard, C.Faivre-Finn, E. Lim, G. Rocco, S. Senan, P. Van Schil, G. Veronesi, R. Stahel, S. Peters, E. F. Felip and Panel Members, Ann. Oncol. 2014, 25, 1462.

Non Patent Literature 3: D. Asanuma, M. Sakabe, M. Kamiya, K. Yamamoto, J. Hiratake, M. Ogawa, N. Kosaka, P. L. Choyke, T. Nagano, H. Kobayashi, Y. Urano, Nat. Commun. 2015, 6, 6463.

Non Patent Literature 4: A. Minami, T. Otsubo, D. Ieno, K. Ikeda, H. Kanazawa, K. Shimizu, K. Ohata, T. Yokochi, Y. Horii, H. Fukmoto, R. Taguchi, T. Takahashi, N. Oku, T. Suzuki, PLoS One, 2014, 9, e 81941.

### SUMMARY OF INVENTION

### Technical Problem to be Solved by the Invention

An object of the present invention is to provide a novel fluorescent probe for sialidase activity detection.

### Means for Solving the Problem

Sialic acids are an acidic monosaccharide that terminates a cell surface glycan, and removal of a sialic acid is catalyzed by a sialidase. It is known that expression of a sialidase is increased in various cancers, and the present inventors examined whether an HMRef-sialic acid complex can be used as a fluorescent probe for sialidase activity detection.

The present inventors have recently developed HMRef-βGal as a probe for β-galactosidase. The present inventors initially substituted the β-galactoside moiety of the sialic acid of HMRef-βGal (Neu5Ac) to synthesize HMRef-Neu5Ac, which is a novel fluorescent probe for a sialidase, and examined the optical property thereof.

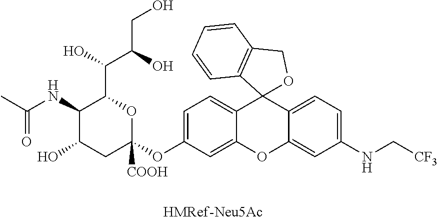 [SMILES: CC(=O)N[C@H]1[C@H]([C@H](O)[C@H](O)CO)O[C@@](Oc2ccc3c(c2)Oc2cc(NCC(F)(F)F)ccc2C32OCc3ccccc32)(C(=O)O)C[C@@H]1O]

However, it has been found that although HMRef-Neu5Ac exhibits non-fluorescent to highly fluorescent properties, this probe exhibits instability over time. The present inventors examined the cause, and found that the glycosidic bond in the sialic acid is very sensitive to acid hydrolysis. The present inventors intensively studied, and as a result, have found that it is possible to suppress acid hydrolysis of the glycosidic bond in the sialic acid by introducing a spacer (self-immolative linker) having a specific structure between the sialic acid (Neu5Ac) moiety and the skeletal structure of HMRef, and provide a probe capable of effectively detecting the sialidase activity, and thus have completed the present invention.

That is, the present invention provides the following items.
[1] A compound represented by a general formula (I) described below or a salt thereof:

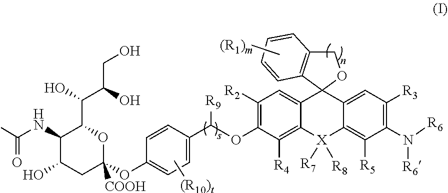

wherein

R₁, if present, represents the same or different monovalent substituent present on a benzene ring;

R₂ and R₃ each independently represent a hydrogen atom, an alkyl group having 1 to 6 carbon atoms, or a halogen atom;

R₄ and R₅ each independently represent a hydrogen atom, an alkyl group having 1 to 6 carbon atoms, or a halogen atom;

R₆ represents a hydrogen atom, an alkyl group having 1 to 5 carbon atoms, or a fluorinated alkyl group having 1 to 5 carbon atoms;

R₆′ represents a hydrogen atom or an alkyl group having 1 to 6 carbon atoms, and

R₆′ may form, together with R₃ or R₅, a five to seven-membered heterocyclyl or heteroaryl containing a nitrogen atom to which R₆′ is bonded;

R₂ and R₈, if present, each independently represent an alkyl group having 1 to 6 carbon atoms or an aryl group, and p in a case where X is an oxygen atom, R₇ and R₈ are not present;

R₉ in each occurrence is independently selected from a hydrogen atom, an alkyl group having 1 to 5 carbon atoms, an alkoxy group, a hydroxyl group, a carboxyl group, a halogen atom, a sulfo group, an amino group, an alkoxycarbonyl group, or an oxo group;

R₁₀, if present, represents the same or different monovalent substituent present on a benzene ring;

X represents an oxygen atom, a silicon atom, or a carbon atom;

m is an integer of 0 to 4;

n is an integer of 1 to 3;

s is an integer of 1; and

t is an integer of 0 to 4.
[2] The compound or a salt thereof according to [1],
wherein R₆ is —CH₂-CF₃.
[3] A fluorescent probe for detecting sialidase activity, comprising the compound or a salt thereof according to [1] or [2].
[4] A method of detecting sialidase activity, the method comprising:

a step (a) of introducing the compound or a salt thereof according to [1] or [2] into a cell; and

a step (b) of measuring fluorescence emitted by the compound or the salt thereof reacted with a sialidase in the cell.

### Advantageous Effects of Invention

The fluorescent probe for detecting sialidase activity of the present invention can be excited by visible light, and emits fluorescence without precipitation of the enzymatic reaction product, and therefore has high biocompatibility. Furthermore, introducing a sialic acid as a substrate via a self-immolative linker gives the fluorescent probe an effect of improving the stability of the compound.

These effects suggest that the probe of the present invention can be applied to various biological and medical studies on sialidases, and the probe is expected to contribute greatly as a research tool for sialidases and a diagnosis tool.

### BRIEF DESCRIPTION OF DRAWINGS

FIG. 1 shows a scheme of enzymatic hydrolysis of HMRef-Neu5Ac and HMRef-S-Neu5Ac as an example of the compound of the present invention in the presence of a sialidase.

FIG. 2a shows changes in fluorescence intensity of HMRef-Neu5Ac over time that are caused by an enzymatic reaction.

FIG. 2b shows results of measuring absorption spectra of HMRef-Neu5Ac at various pH values.

FIG. 3a shows changes in fluorescence intensity of HMRef-S-Neu5Ac over time that are caused by an enzymatic reaction.

FIG. 3b shows results of measuring absorption spectra of HMRef-S-Neu5Ac at various pH values.

FIG. 4 shows results of measuring the fluorescence intensity of HMRef-Neu5Ac and that of HMRef-S-Neu5Ac with time at various pH values.

### DESCRIPTION OF EMBODIMENTS

In the present specification, the term “alkyl group” or an alkyl moiety in a substituent including the alkyl moiety (such as an alkoxy group) means, unless otherwise specified, an alkyl group having, for example, 1 to 6 carbon atoms, preferably 1 to 4 carbon atoms, and more preferably about 1 to 3 carbon atoms that includes a straight chain, a branched chain, a ring, or a combination thereof. More specific examples of the alkyl group include a methyl group, an ethyl group, an n-propyl group, an isopropyl group, a cyclopropyl group, an n-butyl group, a sec-butyl group, an isobutyl group, a tert-butyl group, a cyclopropylmethyl group, an n-pentyl group, and an n-hexyl group.

In the present specification, the term “halogen atom” refers to any one of a fluorine atom, a chlorine atom, a bromine atom, or an iodine atom, and a fluorine atom, a chlorine atom, and a bromine atom are preferable.

One embodiment of the present invention is a compound represented by the following general formula (I) or a salt thereof.

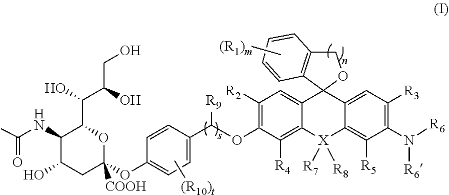

In the present invention, it is important to introduce a spacer (self-immolative linker) having a specific structure between the sialic acid (Neu5Ac) moiety and the skeletal structure of HMRef. By the introduction, it is possible to suppress acid hydrolysis of the glycosidic bond in the sialic acid, and provide a probe capable of effectively detecting the sialidase activity.

In the general formula (I), R₁, if present, represents a monovalent substituent present on a benzene ring, and R₁s are identical or different. Examples of the monovalent substituent include halogens and optionally substituted alkyl groups.

m is an integer of 0 to 4.

In one preferable aspect of the present invention, m is 0, that is, R₁ is absent, and the benzene ring is unsubstituted.

In the general formula (I), R₂ and R₃ each independently represent a hydrogen atom, an alkyl group having 1 to 6 carbon atoms, or a halogen atom.

In a case where R₂ and R₃ represent an alkyl group, one or two or more moieties may be present in the alkyl group, and examples of the moiety include halogen atoms, a carboxy group, a sulfonyl group, a hydroxyl group, an amino group, and an alkoxy group. For example, the alkyl group represented by R₂ or R₃ may be a halogenated alkyl group, a hydroxyalkyl group, a carboxyalkyl group, or the like. It is preferable that R₂ and R₃ be each independently a hydrogen atom or a halogen atom, and it is more preferable that both R₂ and R₃ be a fluorine atom or a chlorine atom.

R₄ and R₅ each independently represent a hydrogen atom, an alkyl group having 1 to 6 carbon atoms, or a halogen atom, and are the same as described above for R₂ and R₃. It is preferable that both R₄ and R₅ be a hydrogen atom.

R₆ represents a hydrogen atom, an alkyl group having 1 to 5 carbon atoms, or a fluorinated alkyl group having 1 to 5 carbon atoms. The alkyl group as R₆ is preferably a methyl group or an ethyl group. The fluorinated alkyl group as R₆ is preferably —CH₂-CF₃ or —CH₂-CH₂-CF₃.

In one preferable aspect of the present invention, R₆ is —CH₂-CF₃.

R₆′ represents a hydrogen atom or an alkyl group having 1 to 6 carbon atoms. In a case where R₆′ represents an alkyl group, one or two or more moieties may be present in the alkyl group, and examples of the moiety include halogen atoms, a carboxy group, a sulfonyl group, a hydroxyl group, an amino group, an alkoxy group and the like. For example, the alkyl group represented by R⁵ or R⁶ may be a halogenated alkyl group, a hydroxyalkyl group, a carboxyalkyl group, or the like.

In one preferable aspect of the compound of the present invention, R6′ is a hydrogen atom.

R₆′ may form, together with R₃ or R₅, a five to seven-membered heterocyclyl or heteroaryl containing a nitrogen atom to which R₆′ is bonded.

The heterocyclyl or heteroaryl may contain, as a ring member, 1 to 3 additional heteroatoms selected from the group consisting of an oxygen atom, a nitrogen atom, and a sulfur atom.

The heterocyclyl or heteroaryl may be substituted with an alkyl group having 1 to 6 carbon atoms, an alkenyl group having 2 to 6 carbon atoms, an alkynyl group having 2 to 6 carbon atoms, an aralkyl group having 6 to 10 carbon atoms (such as a benzyl group or a phenethyl group), or an alkyl-substituted alkenyl group having 6 to 10 carbon atoms.

Examples of the heterocyclyl or heteroaryl thus formed include, but are not limited to, pyrrolidine, piperidine, hexamethyleneimine, pyrrole, imidazole, pyrazole, oxazole, and thiazole.

In the general formula (I), in a case where R₇ and R₈ are present, R₇ and R₈ each independently represent an alkyl group having 1 to 6 carbon atoms or an aryl group, and it is preferable that R₇ and R₈ be each independently an alkyl group having 1 to 3 carbon atoms, and it is more preferable that both R₇ and R₈ be a methyl group. In the alkyl group represented by R₇ and R₈, one or two or more moieties may be present, and examples of the moiety include halogen atoms, a carboxy group, a sulfonyl group, a hydroxyl group, an amino group, an alkoxy group and the like. For example, the alkyl group represented by R₇ or R₈ may be a halogenated alkyl group, a hydroxyalkyl group, a carboxyalkyl group, or the like.

In a case where R₇ or R₈ represents an aryl group, the aryl group may be either a monocyclic aromatic group or a fused aromatic group, and the aryl ring may include one or two or more ring-constituting heteroatoms (such as a nitrogen atom, an oxygen atom, and a sulfur atom). The aryl group is preferably a phenyl group. On the aryl ring, one or two or more substituents may be present. Examples of the substituent include halogen atoms, a carboxy group, a sulfonyl group, a hydroxyl group, an amino group, an alkoxy group and the like, and one or two or more of such substituents may be present.

In a case where X described below is an oxygen atom, R₇ and R₈ are absent.

R₉ in each occurrence is independently selected from a hydrogen atom, an alkyl group having 1 to 5 carbon atoms, an alkoxy group, a hydroxyl group, a carboxyl group, a halogen atom, a sulfo group, an amino group, an alkoxycarbonyl group, or an oxo group, and is preferably a hydrogen atom.

s is an integer of 1.

In a case where R₁₀ is present, R₁₀ represents the same or different monovalent substituent present on a benzene ring.

Examples of the monovalent substituent include halogens and optionally substituted alkyl groups.

t is an integer of 0 to 4.

In one preferable aspect of the present invention, t is 0, that is, R₁₀ is absent, and the benzene ring is unsubstituted.

X represents an oxygen atom, a silicon atom, or a carbon atom.

In one preferable aspect of the present invention, X is an oxygen atom.

n is an integer of 1 to 3, and is preferably 1.

The compound represented by the general formula (I) of the present invention can be present as an acid addition salt or a base addition salt. Examples of the acid addition salt include mineral acid salts such as hydrochlorides, sulfates, and nitrates, and organic acid salts such as methanesulfonates, p-toluenesulfonates, oxalates, citrates, and tartrates, and examples of the base addition salt include metal salts such as sodium salts, potassium salts, calcium salts, and magnesium salts, ammonium salts, and organic amine salts such as triethylamine salts. In addition to these salts, a salt with an amino acid such as glycine is formed in some cases. The compound or a salt of the compound of the present invention is present as a hydrate or a solvate in some cases, and such a substance is also within the scope of the present invention.

The compound represented by the general formula (I) of the present invention has one or two or more asymmetric carbons in some cases according to the kind of the substituent, and all the substances are included in the scope of the present invention such as the stereoisomers such as the optically active substances based on the one or two or more asymmetric carbons and the diastereoisomers based on the two or more asymmetric carbons, mixtures of the stereoisomers, and the racemates.

A method of producing a representative compound of the compound of the present invention is specifically shown in Examples in the present description. Therefore, a person skilled in the art can produce the compound represented by the general formula (I) of the present invention by appropriately selecting a reaction raw material, a reaction condition, a reaction reagent, and the like on the basis of the description in Examples, and modifying or changing the method if necessary.

The compound of the present invention represented by the general formula (I) of the present invention is useful as a fluorescent probe for detecting sialidase activity.

That is, another embodiment of the present invention is a fluorescent probe comprising the compound represented by the general formula (I) or a salt of the compound.

Another embodiment of the present invention is a method of detecting intracellular sialidase activity, and the method comprises a step (a) of introducing the compound represented by the general formula (I) or a salt thereof into a cell, and a step (b) of measuring fluorescence emitted by the compound or the salt thereof reacted with a sialidase in the cell.

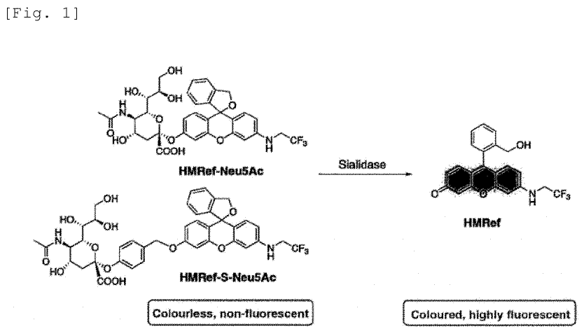

The compound represented by the general formula (I) of the present invention or a salt thereof is characterized by being substantially non-fluorescent or having only weak fluorescence in an environment without a sialidase, and emitting strong fluorescence in an environment with a sialidase. FIG. 1 shows a scheme of enzymatic hydrolysis of HMRef-Neu5Ac and HMRef-S-Neu5Ac as an example of the compound of the present invention in the presence of a sialidase.

Examples of the sialidase that can be detected by the fluorescent probe of the present invention include various sialidases such as sialidases present in a mammalian cell (NEU1, NEU2, NEU3, and NEU4) and bacterial sialidases, but are not limited thereto.

The method of using the fluorescent probe of the present invention is not particularly limited, and the fluorescent probe can be used in the same manner as a conventionally known fluorescent probe. A fluorescence spectrum may be usually measured with the following procedure. The compound represented by the above-described formula (I) or a salt thereof is dissolved in, for example, an aqueous medium such as physiological saline or a buffer, or a mixture of a water-miscible organic solvent such as ethanol, acetone, ethylene glycol, dimethyl sulfoxide, or dimethylformamide and an aqueous medium, and the resulting solution is added to an appropriate buffer containing a cell or a tissue, and the fluorescence spectrum is measured. The fluorescent probe of the present invention may be used in the form of a composition in combination with an appropriate additive. For example, the fluorescent probe can be combined with an additive such as a buffering agent, a solubilizing agent, or a pH adjusting agent.

### EXAMPLES

Hereinafter, the present invention will be described with reference to Examples, but the present invention is not limited thereto.

### [Raw Material]

All the chemical substances used in synthesis were purchased from Tokyo Chemical Industry Co., Ltd., Wako Pure Chemical Industries, Ltd., and Sigma-Aldrich Co. LLC. The chemical substances were used without further purification.

### [Measuring Apparatus]

Nuclear magnetic resonance (NMR) spectra were obtained with a spectrometer, Bruker NMR AVANCE III 400 [1H (400 MHz), 13C (101 MHz)] using a deuterated solvent.

High-resolution electrospray ionization (ESI) mass spectra were obtained with microOTOF II (Bruker).

HPLC purification was performed with a pump, JASCO PU-2087 Plus (GL Science Co., Ltd.) equipped with Inertstil-ODS-3 columns (0 10×250 mm (semi-preparative) and Φ 20×250 mm (preparative)), and with a detector, UVIDEC-100-V (JASCO).

The solvent used for HPLC was obtained from Wako Pure Chemical Industries, Ltd. Silica gel column chromatography was performed using silica gel 60N (spherical, neutral, 63 to 210 μm; manufactured by KANTO CHEMICAL CO., INC.).

TLC was performed with a silica gel plate F 254 (0.25 mm (analysis); Merck, AKG).

UV-vis spectra were obtained with a spectrophotometer, Shimadzu UV-2450.

[Optical Property and in vitro Enzymatic Reaction]

Fluorescence spectra were obtained with Hitachi F-7000. The absolute fluorescence quantum efficiency was measured using an absolute PL quantum yield measuring apparatus, Quantuarus-QY (Hamamatsu Photonics K. K.). The probe was dissolved in dimethyl sulfoxide (DMSO, grade for fluorometry, DOJINDO LABORATORIES) to obtain a stock solution.

The optical property of the probe was acquired in a 0.2 M sodium phosphate buffer, a 100 mM NaOAc, 2 mM CaCl₂, pH 6.5 buffer, or a 50 mM NaOAc, 2 mM CaCl₂, 100 μg/mL BSA, pH 7.4 buffer. For an in vitro enzymatic reaction, the probe DMSO stock solution was diluted with a 100 mM NaOAc, 2 mM CaCl₂, pH 7.4 buffer so that the final concentration was 1 μM. First, the fluorescence of the solution before adding a sialidase was measured every second (excitation wavelength: 490 nm, emission wavelength: 520 nm). Then, a neuraminidase from Arthrobacter ureafaciens (0.01 U) was used for this assay.

### [Dynamic Parameters of HMRef-Neu5Ac, HMRef-S-Neu5Ac, and 4-MUNANA]

The probe was dissolved in 200 μL (total volume) of 50 mM NaOAc, 2 mM CaCl₂, 100 μg/mL BSA buffer (pH 6.5) at various concentrations. A neuraminidase (from Arthrobacter ureafaciens, 50 μM) was added, and the increase in fluorescence for 60 minutes was evaluated. Concerning the dynamic parameter Vₘₐₓ and Kₘ, the corrected data were calculated using the following formula.

$${V}_{0} = \frac{( {V}_{max} ) X}{{K}_{m} + X}$$

V₀ is the steady state speed of the probe substrate rotation by the neuraminidase, Vₘₐₓ is the maximum speed (mM/min), X is the probe concentration in the reaction mixture (mM), and Kₘ is the Michaelis constant (mM) at which the reaction speed is half of Vₘₐₓ. Formula 1 was fitted to the data using KaleidaGraph 4.5.2 software (Synergy Software).

### [Synthesis Example 1]

Synthesis of HMRef-Neu5Ac

HMRef-Neu5Ac was synthesized in accordance with the procedure for the following reaction scheme.

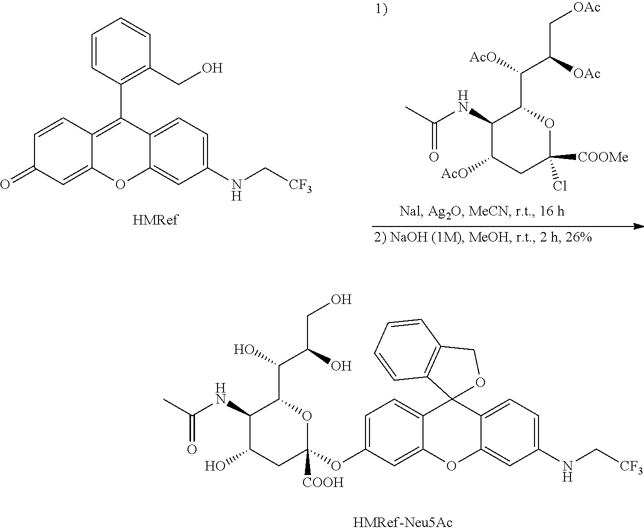 [SMILES: O=c1ccc2c(-c3ccccc3CO)c3ccc(NCC(F)(F)F)cc3oc-2c1 ; COC(=O)[C@@]1(Cl)C[C@H](OC(C)=O)[C@@H](NC(C)=O)[C@H]([C@H](OC(C)=O)[C@@H](COC(C)=O)OC(C)=O)O1 ; CC(=O)N[C@H]1[C@H]([C@H](O)[C@H](O)CO)O[C@@](Oc2ccc3c(c2)Oc2cc(NCC(F)(F)F)ccc2C32OCc3ccccc32)(C(=O)O)C[C@@H]1O]

HMRef was synthesized in accordance with the literature (D. Asanuma, M. Sakabe, M. Kamiya, K. Yamamoto, J. Hiratake, M. Ogawa, N. Kosaka, P. L. Choyke, T. Nagano, H. Kobayashi, Y. Urano, Nat. Commun., 2015, 6, 6463.). N-acetyl-2-chloro-2-dedineuraminic acid methyl ester 4,7,8,9-tetraacetate (393 mg, 0.77 mmol, 6.0 eq.), Ag₂O (488 mg, 2.1 mmol, 16.4 eq.), NaI (150 mg, 1.0 mmol, 7.8 eq.), and a sufficient amount of anhydrous Na₂SO₄ were put into a flask, and HMRef (51.2 mg, 0.13 mmol, 1.0 eq.) dissolved in 20 mL of dry acetonitrile (MeCN) was added thereto. The reaction mixture was stirred at room temperature for 16 hours and then filtered, and the filtrate was distilled off under reduced pressure. The residue was dissolved in methanol (MeOH) (6 mL), and a 1 M NaOH aqueous solution (3 mL) was added. The mixture was stirred at room temperature for 5 hours, and then the organic solvent was distilled off under reduced pressure. The residual aqueous solution was 7neutralized with 2 M HCl and then lyophilized. The residue was then dissolved in a solution containing MeOH and dichloromethane (DCM) at a ratio of 1:4 and filtered to remove the insoluble salt. The resulting solution was distilled off under reduced pressure, and the residue was purified with HPLC (solution A: 100 mM TEAA buffer, solution B: CH₃CN 99% and H₂O 1%, for 5 minutes at A/B=90/10 followed by changing the gradient to 10/90 over 15 minutes, then for 15 minutes at 10/90) and lyophilized to obtain 28 mg of orange powder (26% over 2 steps).

¹H NMR (400 MHz, CD₃OD) δ1.04 (t, 18H, TEAA), 1.86 (1H, t, ³J_{HH}=11.2 Hz, H3ₐₓ) , 1.90 (s, 3H, TEAA) , 2.02 (s, 3H, NHCOCH₃) , 2.55 (q, 12 H, TEAA) , 2.95 (dd, 1H, ³J_{HH}=12.6, 4.4 Hz, H3_{eq}), 3.35 (s, 6H, TEAA), 3.56 (dd, 1H, ³J_{HH}=9.0, 1.6 Hz, H7), 3.67 (dd, 1H, ³J_{HH}=11.3, 5.2 Hz, H9), 3.74 (m, 1H, H5), 3.76 (m, 1H, H4), 3.82 (m, 1H, H9′), 3.84 (m, 2H, NHCH₂CF₃) , 3.87 (m, 1H, H8) , 3.90 (m, 1H, H6) , 5.22 (s, 2H, ArCH₂O), 6.44 (dd, 1H, ³J_{HH}=8.6, ⁴J_{HH}=2.4 Hz, Ar), 6.48 (d, 1H, ⁴J_{HH}=1.5 Hz, Ar), 6.67 (dd, 1H, ³J_{HH}=8.6, ⁴J_{HH}=2.6 Hz, Ar), 6.74 (dd, 1H, ³J_{HH}=8.7, ⁴J_{HH}=2.4 Hz, Ar), 6.78 (d, 1H, ³J_{HH}=7.6 Hz, Ar), 6.93 (dt, 1H, ³J_{HH}=8.7, ⁴J_{HH}=2.5 Hz, Ar), 7.07 (dd, 1H, ⁴J_{HH}=5.6, 2.3 Hz, Ar), 7.26 (t, 1H, ³J_{HH}=7.3 Hz, Ar), 7.38 (t, 1H, ³J_{HH}=7.6 Hz, Ar), 7.42 (d, 1H, ³J_{HH}=7.4 Hz, Ar). ¹³C NMR (101 MHz, CD₃OD):δ11.2 (TEAA) 22.9, 24.4 (TEAA), 43.1, 45.8, 49.9 (TEAA), 54.3, 64.0, 69.3, 70.2, 72.6, 73.1, 75.3, 85.5, 99.3, 104.1, 109.7, 110.8, 114.7, 117.8, 118.1, 121.9, 124.8, 126.9 (q, CF3), 129.3, 129.5, 130.2, 130.9, 140.2, 146.0, 150.3, 152.1, 153.0, 157.0, 174.6, 175.6, 180.7 (TEAA). HRMS (ESI⁺) calcd for [M]⁺691.21092; found 691.21104 (−0.1 mDa).

HPLC analysis: A/B=80/20=0/100 (15 min); flow rate=1.0 mL/min. Detected fluorescence wavelength: 520 nm.

### Reference Example 1

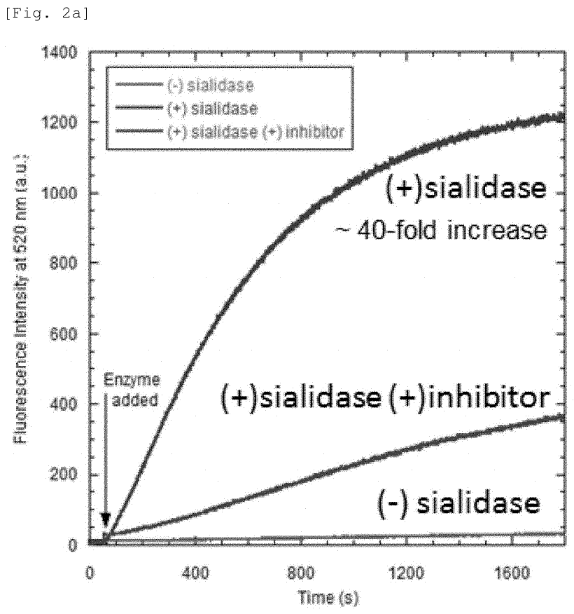

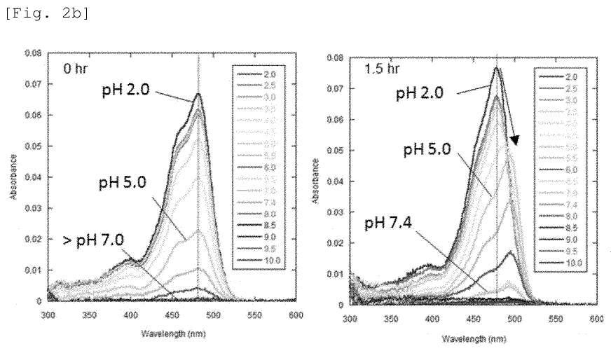

The fluorescence intensity of HMRef-Neu5Ac obtained in Synthesis Example 1 was changed through an enzymatic reaction with a neuraminidase (Arthrobacter ureafaciens), and the change in the fluorescence intensity of HMRef-Neu5Ac over time was examined. HMRef-Neu5Ac was dissolved in a 100 mM NaOAc 2 mM CaCl₂ buffer (pH 7.4) to prepare a 1 μM solution, and 0.01 U of the enzyme was added 60 seconds after the start of measurement. At the time of measurement, the temperature was 37° C., and the concentration of an enzyme inhibitor (DANA) was 100 μM. FIG. 2a shows the results.

HMRef-Neu5Ac was dissolved in 0.2 M sodium phosphate buffers having various pH values, and the absorption spectra were measured immediately after and 1.5 hours after the dissolution. FIG. 2b shows the results.

At pH 7.4, HMRef-Neu5Ac preferentially takes the form of a spiro ring (pKcycl=4.8), and therefore almost no fluorescence was shown. When HMRef-Neu5Ac was reacted with the sialidase, a moderate increase in the fluorescence up to 40-fold was observed (FIG. 2a). In the case of monitoring the identical reaction in the absence of enzyme, a 3-fold increase in the fluorescence was observed, indicating the instability of the probe over time. The absorption spectra of the probe were measured with buffer solutions having various pH values. As a result of comparison between the spectra immediately after the dissolution of the probe and those 1.5 hours after the dissolution, it was found that the spectra 1.5 hours after the dissolution were greatly changed in shape (FIG. 2b). That is, it was found that the absorption maximum shifted during 1.5 hours, that is, changes in shape were observed in the spectra, and the changes clearly showed conversion to HMRef as a hydrolysis product.

The monosaccharide derivative of HMRef previously developed by the present inventors did not show such instability, and therefore it was considered that the sialic acid moiety itself caused the difference in instability. As a result of further investigation, it was found that the glycosidic bond was much more sensitive to acid hydrolysis in sialic acids than in other monosaccharides (J. L. Sonnenburg, H. van Halbeek, A. Varki, J. Biol. Chem., 2002, 277, 17502.). The mechanism of acid hydrolysis of a sialic acid residue is protonation of the ketoside oxygen atom and decomposition into a carbonium oxonium ion resonance-stabilized with an alcohol. The ion reacts with water to produce free saccharide. The protonation of the glycosidic oxygen is promoted when an ionized carboxyl group, which stabilizes the carbonium ion of the intermediate, is in the proximity. Therefore, the inherent instability of the sialic acid residue is induced by the adjacent carboxylic acid.

In order to deal with this fact and prevent stabilization of the carbonium ion, substitution of the carboxyl group was examined. In this strategy, which was previously studied by Chargaff et al., the carboxylic acid moiety was converted to a methyl ester. The glycosidic bond in this derivative had very enhanced stability indeed, but in treatment with a sialidase, no free sialic acid was observed. This meant that this strategy of carboxylic acid protection was not available for designing a sialidase sensitive probe.

Next, the present inventors assumed that there was a possibility that incorporation of a spacer group increased the stability of the probe against acid hydrolysis. From successful applications in which a self-immolative spacer was used in a prodrug or a fluorescent sensor, it has been relatively recently found that the use of a self-immolative spacer is effective (A. Alouane, R. Labruere, T. Le Saux, F. Schmidt, L. Jullien, Angew. Chem. Int. Ed., 2015, 54, 7492.). Thus, the phenol spacer group first reported by Phillips et al. was incorporated between the sialic acid moiety and the HMRef fluorophore to obtain HMRef-S-Neu5Ac as shown in Synthesis Example 2 described below.

### Synthesis Example 2

A compound 3 was synthesized in accordance with the procedure for the following reaction scheme.

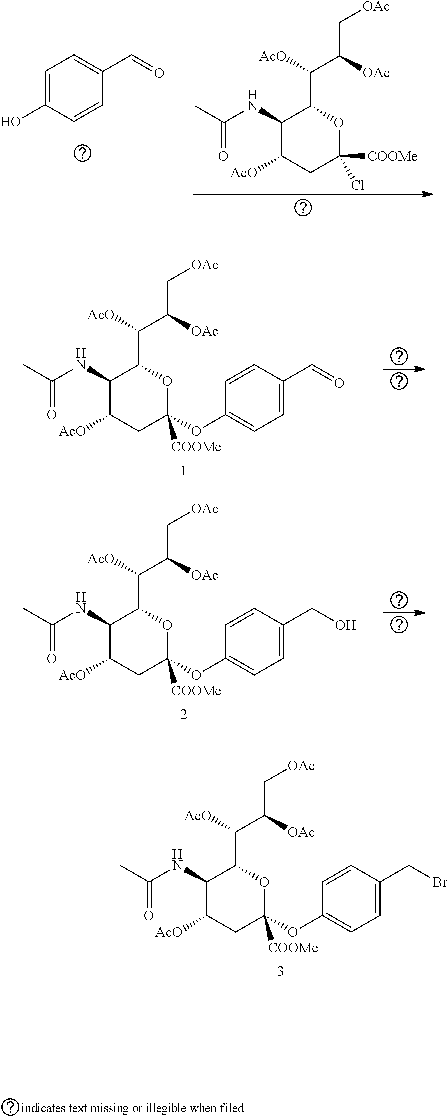 [SMILES: O=Cc1ccc(O)cc1 ; COC(=O)[C@@]1(Cl)C[C@H](OC(C)=O)[C@@H](NC(C)=O)[C@H]([C@H](OC(C)=O)[C@@H](COC(C)=O)OC(C)=O)O1 ; COC(=O)[C@@]1(Oc2ccc(C=O)cc2)C[C@H](OC(C)=O)[C@@H](NC(C)=O)[C@H]([C@H](OC(C)=O)[C@@H](COC(C)=O)OC(C)=O)O1 ; COC(=O)[C@@]1(Oc2ccc(CO)cc2)C[C@H](OC(C)=O)[C@@H](NC(C)=O)[C@H]([C@H](OC(C)=O)[C@@H](COC(C)=O)OC(C)=O)O1 ; COC(=O)[C@@]1(Oc2ccc(CBr)cc2)C[C@H](OC(C)=O)[C@@H](NC(C)=O)[C@H]([C@H](OC(C)=O)[C@@H](COC(C)=O)OC(C)=O)O1]

(1) Synthesis of Compound 1

To a solution of 4-hydroxybenzaldehyde (S, 479 mg, 3.9 mmol) and diisopropylethylamine (5 mL), an MeCN solution of N-acetyl-2-chloro-2-deoyzneuraminic acid methyl ester 4,7,8,9-tetraacetate 3 (0.5 mL, 3.1 mmol) was added, and the reaction mixture was stirred at room temperature for 3 hours. Then, all the solvent was removed, and the resulting residue was diluted with toluene (×3). The resulting residue was purified using silica gel chromatography (ethyl acetate (EtOAc):DCM, 1:1 to EtOAc) to obtain a compound 1 as white foam (145 mg, 62%).

¹H NMR (400 MHz, CDCl₃):δ1.93 (s, 3H, NHOAc), 2.05 (s, 3H, OAc), 2.06 (s, 3H, OAc), 2.12 (s, 3H, OAc), 2.19 (s, 3H, OAc), 2.30 (t, 1H, ³J_{HH}=12.5 Hz, H3ₐₓ), 2.74 (dd, 1H, ³J_{HH}=13, 4.7 Hz, H3_{eq}), 3.65 (s, 3H, COOMe), 4.11 (m, 1H, H9), 4.13 (m, 1H, H5), 4.25 (dd, 1H, ³J_{HH} =12.4, 2.4 Hz, H9′), 4.60 (dd, 1H, ³J_{HH}=10.8, 1.6 Hz, H6), 4.98 (td, 1H, ³J_{HH}=10.4, 4.6, 1.8 HZ, H4), 5.23 (d, 1H, ³J_{HH}=10 Hz, H7), 5.35 (m, 1H, NH), 5.37 (m, 1H, H8), 7.18 (d, 2H, ³J_{HH}=8.7 Hz, Ar), 7.83 (d, 2H, ³J_{HH}=8.8 Hz, Ar), 9.93 (s, 1H, CHO). ¹³C NMR (101 MHz, CDCl₃):δ20.7, 20.7, 20.8, 21.0, 23.2, 38.7, 49.5, 53.2, 62.0, 67.1, 68.4, 68.7, 73.6, 99.5, 118.9, 131.7, 132.0, 158.9, 168.2, 169.9, 170.1, 170.2, 170.6, 170.9, 190.9. HRMS (ESI⁺):calcd for [M+Na⁺] 618.17933, found 618.18094 (−1.6 mDa) for C₂₇H₃₄NaNO₁₄.

(2) Synthesis of Compound 2

The compound 1 (247 mg, 0.42 mmol) was dissolved in dry tetrahydrofuran (THF) (5 mL), and a 1.0 M THF solution of LiAlH(OtBu)₃ (0.83 mL) was added at 0° C. After stirring at 0° C. for 1 hour, saturated NH₄Cl (aqueous) (5 mL) and EtOAc (10 mL) were added to the mixture, and the resulting mixture was stirred at room temperature for an additional 1 hour. A saturated solution of Rochelle salt (aqueous) (10 mL) was added to the mixture, the crude product was extracted with CHCl₃ (×3), and then the organic layer was distilled off under reduced pressure. The residue was then purified using silica gel chromatography (MeOH:DCM 1:99 to 5:95) to obtain a compound 2 as white foam (156 mg, 63%).

¹H NMR (400 MHz, CDCl₃):δ1.90 (s, 3H, NHOAc) , 2.04 (s, 3H, OAc), 2.04 (s, 3H, OAc), 2.12 (s, 3H, OAc), 2.13 (s, 3H, OAc), 2.21 (t, 1H, ³J_{HH}=12.6 Hz, H3ₐₓ), 2.72 (dd, 1H, ³J_{HH}=12.9, 4.6 Hz, H3_{eq}), 3.68 (s, 3H, COOMe), 4.10 (m, 1H, H5), 4.14 (d, 1H, ³J_{HH}=5 Hz, H9), 4.28 (dd, 1H, ³J_{HH}=12.6, 2.6 Hz, H9′), 4.38 (dd, 1H, ³J_{HH}=10.8, 1.6 Hz, H6), 4.63 (s, 2H, CH₂OH), 4.95 (td, 1H, ³J_{HH}=10.4, 4.6, 1.8 HZ, H4), 5.27 (m, 1H, NH), 5.35 (m, 2H, H7, H8), 7.03 (d, 2H, ³J_{HH}=8.6 Hz, Ar), 7.27 (d, 2H, ³J_{HH}=8.6 Hz, Ar). ¹³C NMR (101 MHz, CDCl₃):δ20.7, 20.8, 20.8, 21.0, 23.2, 38.2, 49.5, 52.9, 62.1, 64.9, 67.4, 68.8, 69.4, 73.4, 99.9, 120.3, 128.3, 136.7, 153.1, 168.1, 170.0, 170.1, 170.3, 170.7, 170.9. HRMS (ESI⁺): calcd for [M+Na⁺] 620.19498, found 620.19672 (−1.7 mDa) C₂₇H₃₆NaNO₁₄.

(3) Synthesis of Compound 3

The compound 2 (150 mg, 0.25 mmol) was dissolved in DCM (3 mL), and phosphorus tribromide (12 μL, 0.13 mmol) was added to the solution at 0° C. After stirring at 0° C. for 2 hours, the solution was washed with a saturated NaHCO₃ aqueous solution (×3) and saturated saline (×1). The organic layer was dried with sodium sulfate, and distilled off under reduced pressure to obtain a compound 3 as white foam (136 mg, 82%).

¹H NMR (400 MHz, CDCl₃):δ1.84 (s, 3H, NHOAc), 1.97 (s, 3H, OAc), 1.98 (s, 3H, OAc), 2.05 (s, 3H, OAc), 2.09 (s, 3H, OAc), 2.15 (t, 1H, ³J_{HH}=12.6 Hz, H3ax), 2.63 (dd, 1H, ³J_{HH}=13, 4.7 Hz, H3eq), 3.58 (s, 3H, COOMe), 4.03 (m, 1H, H9), 4.10 (m, 1H, H5), 4.23 (m, 1H, H9′), 4.37 (m, 1H, H6), 4.40 (s, 2H, CH₂Br), 4.89 (td, 1H, ³J_{HH}=10.4, 4.6, 1.8 Hz, H4), 5.29 (m, 3H, H7, H8, NH), 6.95 (d, 2H, ³J_{HH}=8.7 Hz, Ar), 7.23 (d, 2H, ³J_{HH}=8.7 Hz, Ar). ¹³C NMR (101 MHz, CDCl₃):δ19.7, 19.8, 19.8, 20, 22.2, 32.3, 37.2, 48.4, 52.0, 70.0, 66.3, 67.7, 68.1, 72.4, 98.8, 118.7, 129.2, 132.1, 152.8, 167.1, 169.0, 169.1, 169.2, 169.6, 169.9. HRMS (ESI⁺): calcd for [M+Na⁺] 682.11057, found 682.10666 (3.9 mDa) C₂₇H₃₄BrNNaO₁₃.

(4) Synthesis of HMRef-S-Neu5Ac

HMRef-S-Neu5Ac was synthesized in accordance with the procedure for the following reaction scheme.

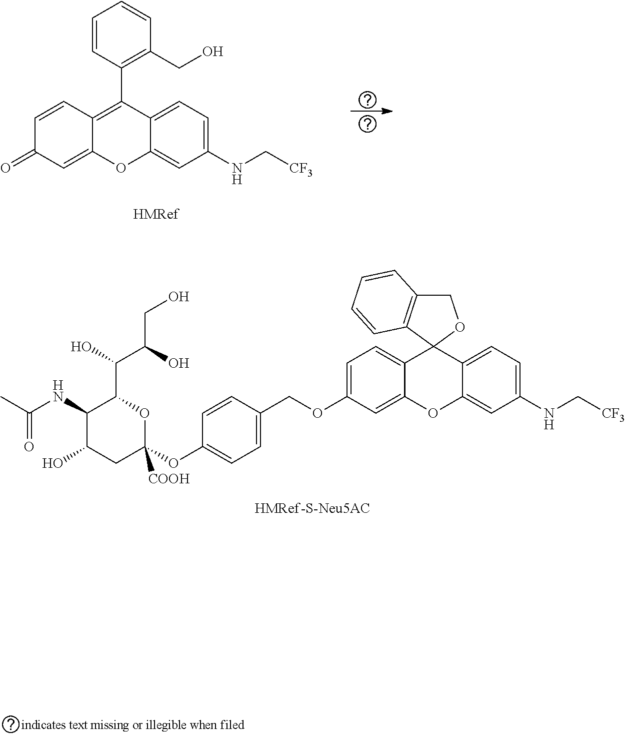 [SMILES: O=c1ccc2c(-c3ccccc3CO)c3ccc(NCC(F)(F)F)cc3oc-2c1 ; CC(=O)N[C@H]1[C@H]([C@H](O)[C@H](O)CO)O[C@@](Oc2ccc(COc3ccc4c(c3)Oc3cc(NCC(F)(F)F)ccc3C43OCc4ccccc43)cc2)(C(=O)O)C[C@@H]1O]

To HMRef (79 mg, 0.2 mmol) and NaH (6 mg, 0.26 mmol), dry THF (3 mL) and dry DMF (1 mL) were added, and the resulting mixture was stirred at 0° C. under an argon atmosphere. A dry THF (3 mL) solution of the compound 3 (130 mg, 0.2 mmol) was added, and the reaction mixture was stirred at room temperature for 16 hours. Then, EtOAc (20 mL) was added, and the resulting mixture was washed with a saturated NH₄Cl solution (×2) and saturated saline (×1). The organic layer was dried over sodium sulfate and distilled off under reduced pressure. The residue was dissolved in MeOH (6 mL), and 1 M NaOH (aqueous solution) (3 mL) was added. The mixture was stirred at room temperature for 5 hours, and then the organic solvent was evaporated. The residual aqueous solution was neutralized with 2 M HCl and then lyophilized. The residue was then dissolved in a solution containing MeOH and DCM at a ratio of 1:4 and filtered to remove the insoluble salt. The resulting solution was distilled off under reduced pressure, and the residue was purified with HPLC (solution A: 100 mM TEAA buffer, solution B: CH₃CN 99% and H₂O 1%, for 5 minutes at A/B=90/10 followed by changing the gradient to 10/90 over 15 minutes, then for 15 minutes at 10/90) and lyophilized to obtain orange powder (36% over 2 steps).

¹H NMR (400 MHz, CD₃OD) δ1.04 (t, 26H, TEAA), 1.84 (1H, t, ³J_{HH}=11.9 Hz, H3ₐₓ) , 1.90 (s, 3H, TEAA) , 2.02 (s, 3H, NHCOCH₃) , 2.55 (q, 17 H, TEAA) , 2.94 (dd, 1H, ³J_{HH}=12.2, 4.0 Hz, H3_{eq}) , 3.54 (dd, 1H, ³J_{HH}=9.0, 1.6 Hz, H7), 3.66 (dd, 1H, ³J_{HH}=11.3, 5.4 Hz, H9), 3.74 (m, 1H, H5), 3.77 (m, 1H, H4), 3.81 (m, 1H, H9′), 3.85 (m, 2H, NHCH₂CF₃), 3.86 (m, 1H, H8), 3.91 (m, 1H, H6), 5.01 (s, 2H, ArCH₂O), 5.23 (s, 2H, ArCH₂O), 6.45 (dd, 1H, ³J_{HH}=8.6, ⁴J_{HH}=2.4 Hz, Ar), 6.50 (d, 1H, ⁴J_{HH}=2.3 Hz, Ar), 6.65 (dd, 1H, ³J_{HH}=8.7, ⁴J_{HH}=2.6 Hz, Ar), 6.67 (d, 1H, ³J_{HH}=8.6, Ar), 6.78 (m, 1H, Ar), 6.79 (m, 1H, Ar), 6.82 (m, 1H, Ar), 7.24 (d, 2H, ³J_{HH}=8.7 Hz, Ar), 7.30 (m, 1H, Ar), 7.31 (d, 2H, ³J_{HH}=8.7 Hz, Ar), 7.39 (m, 1H, Ar), 7.43 (m, 1H, Ar). ¹³C NMR (101 MHz, CD₃OD):δ11.2 (TEAA) 22.9, 24.4 (TEAA), 43.1, 45.8, 46.9 (TEAA), 54.3, 64.0, 69.3, 70.2, 72.6, 72.7, 73.1, 75.3, 85.5, 99.3, 104.0, 104.1, 109.7, 110.8, 114.7, 117.8, 118.1, 120.8, 121.9, 124.7, 124.8, 126.9 (q, CF₃), 129.3, 129.4, 130.2, 130.2, 130.9, 140.3, 146.0, 150.3, 152.1, 153.0, 157.0, 173.6, 175.6, 180.7 (TEAA). HRMS (ESI⁺) L calcd for [M+Na]⁺797.75163; found 797.75043 (−1.2 mDa).

HPLC analysis was performed using a linear gradient (0 min, 20% CH₃CN/0.1% TFA aq. to 15 min, 100% CH₃CN 0.1% TFA aq; flow rate =1.0 mL/min). Fluorescence at 520 nm was examined.

### Example 1

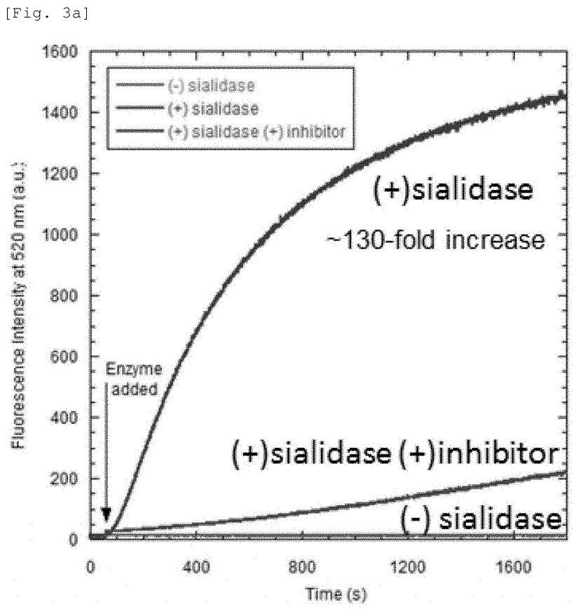

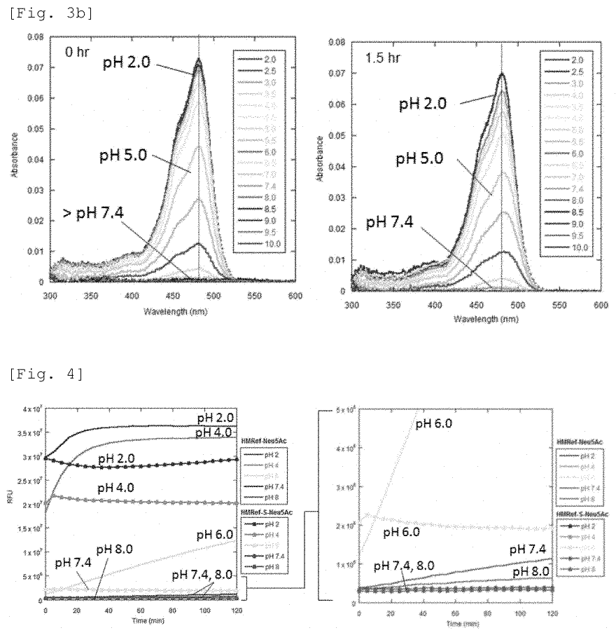

In the same manner as in the evaluation of HMRef-Neu5Ac, the reaction with a sialidase and the absorption spectrum at each pH were acquired for HMRef-S-Neu5Ac (FIGS. 3a and 3b).

At pH 7.4, the probe exists in the form of a spiro ring (pKcycl=5.3), and therefore the fluorescence shown by the probe was very slight. However, the probe showed a 130-fold increase in the fluorescence in reaction with the sialidase, and this is mainly because the stability of the probe was increased. As seen in the case of adding no enzyme, the fluorescence intensity remained at a constant low level, and it was found that the absorption spectra changed little even 1.5 hours after the probe dissolution.

### Example 2

The reactivities of HMRef-Neu5Ac and HMRef-S-Neu5Ac with a sialidase in the presence of a sialidase inhibitor DANA were also evaluated.

Table 1 shows the photochemical properties of HMRef-Neu5Ac and HMRef-S-Neu5Ac. Both HMRef-Neu5Ac and HMRef-S-Neu5Ac show low fluorescence intensity values at pH 7.4. This fact indicates that although HMRef-S-Neu5Ac has more excellent stability, both of the probes function as a sialidase activated fluorescent probe.

TABLE 1

| | Absorption max. (nm) | | Emission max. (nm) | | fluorescence quantum yield^{c} | | | |
|---|---|---|---|---|---|---|---|---|
| | pH 2^{a} | pH 7.4^{b} | pH 2^{a} | pH 7.4^{b} | pH 2^{a} | pH 7.4^{b} | pKₐ | pK_{cycl} |
| HMRef [symbol] | 479 | 498 | 515 | 518 | 0.716 | 0.777 | 4.4 | 10.2 |
| HMRef-Neu5Ac | 484 | — | 522 | — | 0.650 | 0.323 | — | 4.8 |
| HMRef-S-Neu5Ac | 484 | — | 522 | — | 0.537 | 0.271 | — | 5.3 |

^{a}Measurement was performed in 0.2M sodium phosphate buffer (pH 2.0).
^{b}Measurement was performed in 0.2M sodium phosphate buffer at pH 7.4.
^{c}Absolute fluorescence quantum efficiency [symbol]
indicates data missing or illegible when filed

### Example 3

The stability of HMRef-Neu5Ac and that of HMRef-S-Neu5Ac were directly compared by recording the changes in fluorescence intensity over time at a pH within a range. The initial fluorescence intensity values of both the probes depend on the pH value according to the pKcycl values of the probes. That is, the lower the pH is, the larger the initial fluorescence intensity value is.

FIG. 4 shows results of measuring the fluorescence intensity of HMRef-Neu5Ac and that of HMRef-S-Neu5Ac with time at various pH values. The measurement was performed in 0.2 M sodium phosphate buffer at 37° C. and measurements were taken every 5 minutes. In each measurement, excitation/emission wavelengths of 485 nm/535 nm were used.

As can be seen from FIG. 4, HMRef-Neu5Ac showed clear instability at all the pH values over the 2-hour time range of the experiment. At pH 2 and pH 4, the instability was particularly significant, and complete hydrolysis was observed after about 40 minutes. Meanwhile, the fluorescence intensity of HMRef-S-Neu5Ac changed little over the time to confirm its improved stability for 2 hours. The optimal pH for efficient catalysis by mammalian sialidases is in a broad range of 4.4 to 6.5 (T. Miyagi, K. Yamaguchi, Glycobiology, 2012, 22, 880.). Therefore, determining the stability of the probe over these relevant pH values is critical, and it was confirmed that HMRef-S-Neu5Ac showed no increase in fluorescence intensity due to acid hydrolysis.

The dynamic parameters of HMRef-Neu5Ac and HMRef-S-Neu5Ac were determined in accordance with the method previously described by Govorkova et al., and compared to that of a commercial sialidase substrate, 4-MUNANA. The k_{cat}/Kₘ values (Table 2) of HMRef-Neu5Ac and HMRef-S-Neu5Ac were nearly 16 and 13 fold larger than that of 4-MUNANA respectively, demonstrating that the novel probe is further suitable for sensitivity detection and quantitative detection of sialidase activity.

TABLE 2

| | 4-MUNANA | HMRef-Neu5Ac | HMRef-S-Neu5Ac |
|---|---|---|---|
| Kₘ (μM) | 37 | 19 | 11 |
| Vₘₐₓ (nMs⁻¹) | 1.73 | 13.5 | 7.03 |
| k_{cat} (s⁻¹) | 2.6 | 20.6 | 10.7 |
| k_{cat}/Kₘ (μM⁻¹s⁻¹ | 0.07 | 1.1 | 0.9 |

This study targeted sialidases, which are the majority of a group of enzymes known to cleave the final sialic acid from glycoproteins and glycoconjugates on glycolipids, as targeted enzymes. HMRef-Neu5Ac, the first probe developed to detect a sialidase, was shown to be unstable because the carboxyl acid moiety of the sialic acid residue was adjacent to the glycosidic bond. This adjacent carboxyl group stabilizes the intermediate carbonium ion to promote the acid hydrolysis mechanism. The present inventors introduced a spacer group between the sialic acid and the HMRef fluorophore to obtain HMRef-S-Neu5Ac, and thus have achieved reduction in efficiency of the undesirable hydrolysis pathway, resulting in improvement in the stability of the probe.

1. A compound represented by a general formula (I) described below or a salt thereof: 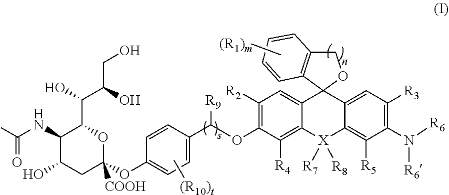
wherein
R₁, if present, represents the same or different monovalent substituent present on a benzene ring;
R₂ and R₃ each independently represent a hydrogen atom, an alkyl group having 1 to 6 carbon atoms, or a halogen atom;
R₄ and R₅ each independently represent a hydrogen atom, an alkyl group having 1 to 6 carbon atoms, or a halogen atom;
R₆ represents a hydrogen atom, an alkyl group having 1 to 5 carbon atoms, or a fluorinated alkyl group having 1 to 5 carbon atoms;
R₆′ represents a hydrogen atom or an alkyl group having 1 to 6 carbon atoms, and
R₆′ may form, together with R₃ or R₅, a five to seven-membered heterocyclyl or heteroaryl containing a nitrogen atom to which R₆′ is bonded;
R₇ and R₈, if present, each independently represent an alkyl group having 1 to 6 carbon atoms or an aryl group, and
in a case where X is an oxygen atom, R₇ and R₈ are not present;
R₉ in each occurrence is independently selected from a hydrogen atom, an alkyl group having 1 to 5 carbon atoms, an alkoxy group, a hydroxyl group, a carboxyl group, a halogen atom, a sulfo group, an amino group, an alkoxycarbonyl group, or an oxo group;
in a case where R₁₀ is present, R₁₀ represents the same or different monovalent substituent present on a benzene ring;
X represents an oxygen atom, a silicon atom, or a carbon atom;
m is an integer of 0 to 4;
n is an integer of 1 to 3;
s is an integer of 1; and
t is an integer of 0 to 4.

2. The compound or a salt thereof according to claim 1, wherein R₆ is —CH₂-CF₃.

3. A fluorescent probe for sialidase activity detection, comprising the compound or a salt thereof according to claim 1.

4. A method of detecting sialidase activity, the method comprising:
introducing the compound or a salt thereof according to claim 1 into a cell; and
measuring fluorescence emitted by the compound or the salt of the compound reacted with a sialidase in the cell.
